(19) United States
(12) Patent Application Publication
(10) Pub. No.: US 2026/0137566 A1
(43) Pub. Date: May 21, 2026

(54) CONTAINER, WOUND DRESSING ASSEMBLY, AND WOUND DRESSING WITH OPTICAL PATTERNS

(71) Applicant: Solventum Intellectual Properties Company, Maplewood, MN (US)

(72) Inventors: Jonathan D. Gandrud, Woodbury, MN (US); Audrey A. Sherman, Woodbury, MN (US); Marie D. Manner, St. Paul, MN (US)

(73) Assignee: Solventum Intellectual Properties Company, Maplewood, MN (US)

(21) Appl. No.: 19/365,424

(22) Filed: Oct. 22, 2025

Related U.S. Application Data
(60) Provisional application No. 63/711,055, filed on Oct. 23, 2024.

(51) Int. Cl. A61F 15/00

(52) U.S. Cl. CPC A61F 15/001

(57) ABSTRACT

A container, a wound dressing, and a wound dressing assembly are disclosed. The container may include one or more optical patterns that facilitate determining whether the container has been tampered with using an optical reader. The wound dressing may include one or more optical patterns that facilitate determining whether a stretchable substrate thereof is in a correctly-tensioned state using the optical reader. The wound dressing assembly may include multiple optical patterns that facilitate determining whether a cover of the wound dressing assembly is correctly positioned on a wound dressing of the wound dressing assembly using the optical reader.

### CROSS REFERENCE TO RELATED APPLICATIONS

This application claims the benefit of U.S. Provisional Application No. 63/711,055, filed Oct. 23, 2024, the disclosure of which is incorporated by reference herein in its entirety.

### TECHNICAL FIELD

The present disclosure relates generally to a container for a medical device, a wound dressing assembly, and a wound dressing including optical patterns.

### BACKGROUND

Various medical articles may house and restrict access to medical devices. For example, containers may be designed to restrict unauthorized access to intravenous devices, since unauthorized access to the intravenous devices may lead to infection and/or introduction of forbidden substances into intravenous lines via ports of the intravenous devices. Therefore, it may be important to detect unauthorized access to the medical devices (i.e., tampering). Furthermore, it may be important to maintain records of the events that occur during use of the containers for safety and compliance purposes. Moreover, various medical articles, such as dressings, may include a stretchable substrate. The stretchable substrate may need to be correctly-tensioned for optimal use of the dressings. Therefore, it may be important to detect whether the stretchable substrate is correctly-tensioned during use of the dressings.

### SUMMARY

In a first aspect, the present disclosure provides a container for a medical device. The container includes a base for holding the medical device. The container further includes a cover configured to be coupled with the base. The container further includes a locking piece for locking the cover to the base. The container further includes multiple optical patterns disposed on at least one of the base, the cover, or the locking piece. When the container is closed and the locking piece is locked, the multiple optical patterns form a combined optical pattern readable by an optical reader to obtain a first information. When the container is opened and unlocked after being closed and locked, the multiple optical patterns are unreadable by the optical reader.

In a second aspect, the present disclosure provides a wound dressing assembly. The wound dressing assembly includes a wound dressing. The wound dressing assembly further includes a cover configured to be attached to the wound dressing. The wound dressing assembly further includes multiple optical patterns disposed on at least one of the wound dressing or the cover. When the cover is correctly attached to the wound dressing, the multiple optical patterns form a combined optical pattern that is readable by an optical reader to obtain a first information. When the cover is incorrectly attached to or detached from the wound dressing, the multiple optical patterns are unreadable by the optical reader

In a third aspect, the present disclosure provides container for a medical device. The container includes a base for holding the medical device. The container further includes a cover configured to be coupled to the base. The container further includes a locking piece for locking the cover to the base. The container further includes an optical pattern disposed or disposable on at least one of the base, the cover, or the locking piece. The optical pattern encodes a first information. When the container is closed and the locking piece is locked, the optical pattern is readable by an optical reader to obtain the first information. When the container is opened and unlocked after being closed and locked, the optical pattern is modified to a modified optical pattern. The modified optical pattern has at least one characteristic different from the optical pattern.

In a fourth aspect, the present disclosure provides a wound dressing. The wound dressing includes a stretchable substrate. The stretchable substrate includes an under-tensioned state, a correctly-tensioned state, and an over-tensioned state. The stretchable substrate is configured to be transitioned between the under-tensioned state, the correctly-tensioned state, and the over-tensioned state. The wound dressing further includes a first optical pattern disposed on the stretchable substrate. The first optical pattern encodes a first information. The first optical pattern is readable by an optical reader in the correctly-tensioned state of the stretchable substrate to obtain the first information. The first optical pattern is unreadable by the optical reader in each of the under-tensioned state and the over-tensioned state of the stretchable substrate.

The details of one or more embodiments of the disclosure are set forth in the accompanying drawings and the description below. Other features, objects, and advantages of the disclosure will be apparent from the description and drawings, and from the claims.

### BRIEF DESCRIPTION OF DRAWINGS

Exemplary embodiments disclosed herein are more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labelled with the same number.

FIG. 1 is a schematic perspective view of a container for a medical device according to an embodiment of the present disclosure;

FIG. 2 is a schematic perspective view of the container of FIG. 1 where the container is closed and a locking piece is unlocked according to an embodiment of the present disclosure;

FIG. 3 is a schematic perspective view of the container of FIG. 1 where the container is closed and the locking piece is locked according to an embodiment of the present disclosure;

FIG. 4 is a schematic perspective view of a wound dressing assembly according to an embodiment of the present disclosure;

FIG. 5 is a schematic perspective view of the wound dressing assembly of FIG. 4 where a cover of the wound dressing assembly is detached from a wound dressing of the wound dressing assembly according to an embodiment of the present disclosure;

FIG. 6 is a schematic perspective view of a container according to another embodiment of the present disclosure where the container is closed and a locking piece is locked;

FIG. 7 is a schematic perspective view of the container of FIG. 6 where the container is closed and the locking piece is unlocked according to an embodiment of the present disclosure;

FIG. 8 is a schematic perspective view of a container for a medical device according to another embodiment of the present disclosure;

FIG. 9 is a schematic perspective view of the container of FIG. 8 where the container is closed and a locking piece is locked according to an embodiment of the present disclosure;

FIG. 10 is a schematic perspective view of the container of FIG. 8 where the container is closed and the locking piece is unlocked according to an embodiment of the present disclosure;

FIG. 11A is a schematic top view of a wound dressing according to an embodiment of the present disclosure with a stretchable substrate thereof in an under-tensioned state;

FIG. 11B is a schematic top view of the wound dressing of FIG. 11A with the stretchable substrate in a correctly-tensioned state according to an embodiment of the present disclosure; and

FIG. 11C is a schematic top view of the wound dressing of FIG. 11A with the stretchable substrate in an over-tensioned state according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, “a,” “an,” “the,” “at least one,” and “one or more” are used interchangeably.

As used herein as a modifier to a property or attribute, the term “generally,” unless otherwise specifically defined, means that the property or attribute would be readily recognizable by a person of ordinary skill but without requiring absolute precision or a perfect match (e.g., within +/−20 % for quantifiable properties).

As used herein, the term “substantially,” unless otherwise specifically defined, means to a high degree of approximation (e.g., within +/−10% for quantifiable properties) but again without requiring absolute precision or a perfect match.

As used herein, all numbers should be considered modified by the term “about.” The term “about,” unless otherwise specifically defined, means to a high degree of approximation (e.g., within +/−5% for quantifiable properties) but again without requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A or B” should be understood to mean “only A, only B, or both A and B.”

As used herein, the term “between about,” unless otherwise specifically defined, generally refers to an inclusive or a closed range. For example, if a parameter X is between about A and B, then A≤X≤B.

Unless specified or limited otherwise, the terms “attached,” “connected,” “coupled,” and variations thereof, are used broadly and encompass both direct and indirect attachments, connections, and couplings.

As used herein, the term “configured to” is at least as restrictive as the term “adapted to” and requires actual design intention to perform the specified function rather than mere physical capability of performing such a function.

As used herein, the term “spaced apart” refers to elements that are disposed at a distance from one another.

As used herein, the term “medical device” refers to any device that is used for medical purposes. Medical devices may include intravenous lines, catheters, fluid supply and drainage lines, connectors, adaptors, and the like, all of which may be at least partially received within and secured by a container of the present disclosure.

As used herein, the term “optical pattern” refers to any graphic representation that can store or encode data or information. One specific example of an optical pattern includes a linear or two-dimensional code, where information is coded by appropriate combinations of elements with a predetermined shape, such as a square, rectangle, or hexagon, of dark colors (normally black), separated by light elements (spaces, normally white), such as bar codes, stacked codes (including PDF417), MAXICODES, DATAMATRIX, QR codes, or color codes, etc. More generally, the term “optical pattern” further includes other graphic forms with an information-storing function, including uncoded printed characters (letters, numbers, etc.) and specific shapes (patterns) (such as stamps, logos, signatures, etc.). In some examples, optical pattern(s) may be invisible to the naked eye. Specifically, the optical pattern(s) may be visible only in certain ranges of the electromagnetic spectrum other than the visible light spectrum. For example, the optical pattern(s) may be visible only in the infrared, the ultraviolet, or other non-visible light spectrum of the electromagnetic spectrum. In another example, the optical patterns may be polarized (e.g., circularly polarized). Circularly polarized optical patterns may be difficult to view with the naked eye but may be readable by an optical reader equipped with polarizer filters and/or under a polarized light (e.g., linearly, or circularly polarized light).

As used herein, the term “information” encoded by an optical pattern refers to any information represented by the optical pattern that is readable by an optical reader. The information may be textual information, such as a text associated with a product (e.g., name, manufacturing date, expiry date, authentication code, etc.), a text associated with a patient (e.g., name, admission date, etc.).

As used herein, the term “optical reader” refers to a device that can read an optical pattern. Optical readers may decode an encoded information from the optical pattern. In other words, optical readers may scan the optical patterns to determine the information encoded by the optical patterns. Optical readers may be communicably connected to a computing device, which may process the information determined by the optical readers. Optical readers may be part of handheld computing devices. Optical readers may also be mounted on an equipment. Optical readers may be equipped with filters (e.g., a polarizer filter).

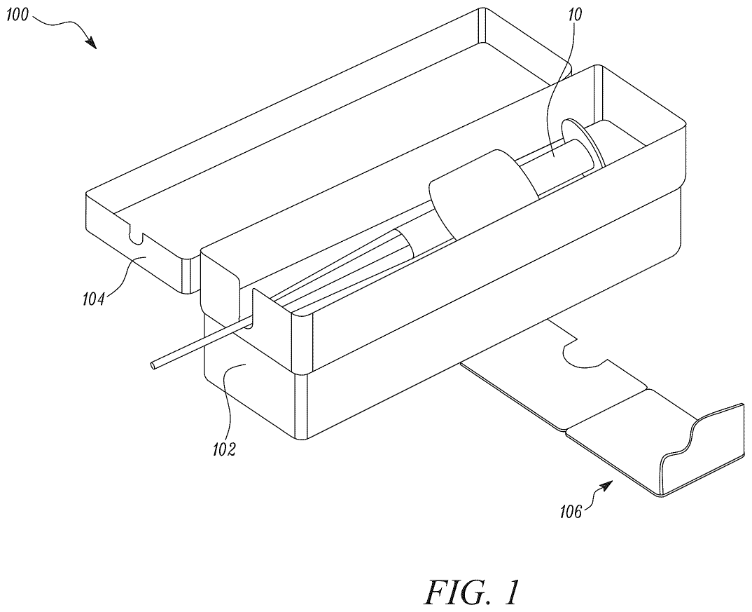

Referring now to the figures, FIGS. 1 to 3 illustrate a container 100 according to an embodiment of the present disclosure.

The container 100 includes a base 102 for holding the medical device 10. The medical device 10 may be any suitable medical device. As an example, the medical device 10 may be an intravenous device. The intravenous device may connect to an end of an intravenous line to administer a drug via the intravenous line. As another example, the medical device 10 may be a needleless connector.

The container 100 further includes a cover 104 configured to be coupled to the base 102. The container 100 further includes a locking piece 106 for locking the cover 104 to the base 102. The locking piece 106 may be configured for locking the cover 104 to the base 102 by using any suitable technique. For example, the locking piece 106 may be configured for locking the cover 104 to the base 102 using a snap-fit connection, an adhesive connection, a friction-fit connection, and so forth.

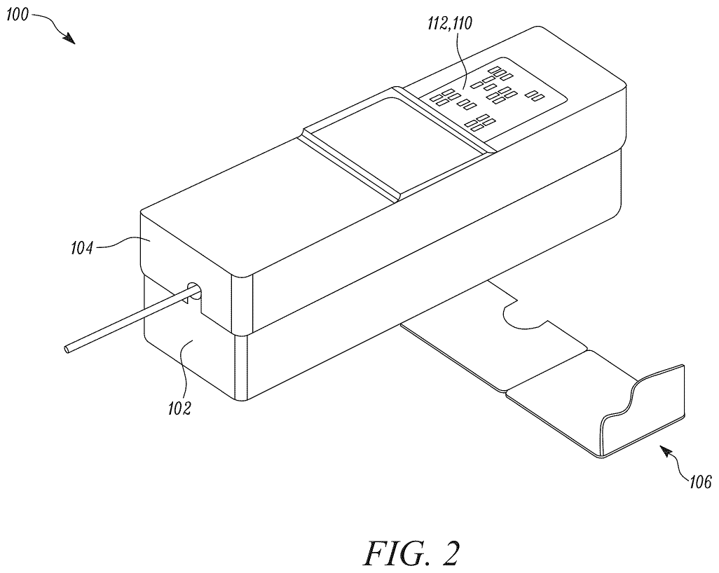

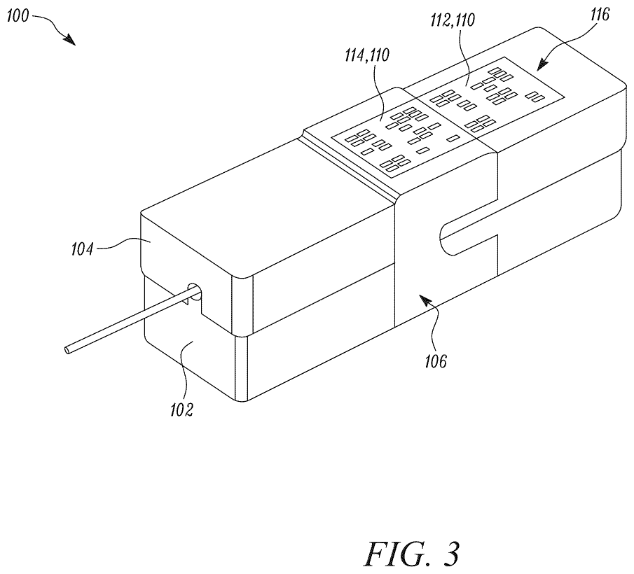

The container 100 further includes multiple optical patterns 110 (shown in FIGS. 2 and 3) disposed on at least one of the base 102, the cover 104, or the locking piece 106. Each optical pattern 110 from the multiple optical patterns 110 may be, for example, a partial optical pattern. In the illustrated embodiment of FIG. 3, the multiple optical patterns 110 include a first optical pattern 112 disposed on the cover 104 and a second optical pattern 114 disposed on the locking piece 106 The first optical pattern 112 may be unreadable by the optical reader by itself. Further, the second optical pattern 114 may be unreadable by the optical reader by itself. In other words, the first optical pattern 112 and the second optical pattern 114 may be unreadable by the optical scanner independently.

When the container 100 is closed (i.e., the cover 104 is coupled to the base 102) and the locking piece 106 is locked (as shown in FIG. 3), the multiple optical patterns 110 form a combined optical pattern 116 readable by an optical reader to obtain a first information. In some embodiments, when the container 100 is closed and the locking piece 106 is locked, the first optical pattern 112 and the second optical pattern 114 may together form the combined optical pattern 116 that is readable by the optical reader to obtain the first information.

When the container 100 is opened and unlocked (as shown in FIG. 2) after being closed and locked, the multiple optical patterns 110 are unreadable by the optical reader. In FIG. 2, the container 100 is depicted as closed (i.e., the cover 104 is coupled to the base 102) and the locking piece 106 is unlocked. In some embodiments, when the container 100 is opened and unlocked after being closed and locked, the first optical pattern 112 and the second optical pattern 114 may be misaligned. In some embodiments, when the container 100 is opened and unlocked after being closed and locked, the first optical pattern 112 and the second optical pattern 114 may be spaced

apart from each other. Therefore, when the container 100 is opened and unlocked after being closed and locked, the optical reader may fail to obtain the first information encoded by the combined optical pattern 116 (shown in FIG. 3). Such a scenario may indicate to an attempt of tampering with the container 100.

In some embodiments, the container 100 may be disposable. Specifically, the container 100 may be designed to be disposed of after a single use. In such embodiments, the multiple optical patterns 110 may form the combined optical pattern 116 only once (for example, the locking piece 106 may lock the cover 104 to the base 102 only once). Therefore, the multiple optical patterns 110 may form the combined optical pattern 116 only once. Subsequent to the single use, the multiple optical patterns 110 may not form the combined optical pattern 116, even after closing the container 100 after the container 100 is opened. Therefore, the container 100 may be conveniently scanned using the optical reader to determine whether the container 100 is tampered or not based on an arrangement of the multiple optical patterns 110. The container 100 may therefore function as a tamper evident device.

The container 100 may also facilitate documenting a series of events during a treatment of a patient. For example, a healthcare practitioner (e.g., a nurse, a medical assistant, a doctor, other clinicians, etc.) may secure the medical device 10 within the container 100 and may perform a first scan of the container 100 using the optical reader. The optical reader obtaining the first information (i.e., successfully reading the combined optical pattern 116) in the first scan may indicate or verify that the container 100 is secured. Various initial record information, such as an image of the patient, an image of the container 100, the date, the time, and the like may be recorded (e.g., in a computing device) subsequent to the first scan. The healthcare practitioner may then proceed with their other duties.

After some time elapses, the healthcare practitioner may return to the patient and perform a second scan of the container 100 using the optical reader. Various final record information, such as an image of the patient, an image of the container 100, the date, the time, and the like may be recorded (e.g., in a computing device) subsequent to the second scan.

As discussed above, upon an attempt of tampering with the container 100, the multiple optical patterns 110 may no longer form the combined optical pattern 116. The optical reader obtaining the first information in the second scan (i.e., successfully reading the combined optical pattern 116) may indicate or verify that the container 100 was not tampered with, and the patient

remained protected in the absence of the healthcare practitioner. The healthcare practitioner may discard the container 100, administer new treatment via the medical device 10, and secure the medical device 10 with another unit of the container 100. However, the optical reader failing to obtain the first information in the second scan (i.e., failing to read the combined optical pattern 116) may indicate potential tampering with the container 100 in the absence of the healthcare practitioner. Evidence of tampering (e.g., the final record information) may be reported in such cases (e.g., reported to the healthcare practitioner's supervisor, or other clinicians). The aforementioned steps may be performed in a cycle.

In some embodiments, the multiple optical patterns 110 may be circularly polarized codes (e.g., circularly polarized optical codes, and the like). A circularly polarized code may not be easily viewable to the naked eye, but may become visible to the optical reader (e.g., a computer vision system) which is equipped with proper filters. For example, a computer vision system may include a quarter-waveplate circular polarizer filter placed in front of a light source. The light source may be used to illuminate the circularly polarized codes (e.g., a code that has been patterned onto the container 100 or a code which has been patterned onto a sticker, a wrap or a decal that is attached to the container 100). Another quarter-waveplate circular polarizer filter may be placed in front of the optical reader (e.g., a digital camera or other optically sensitive device), which is then used to detect the circularly polarized codes.

The circularly polarized codes may not be easily viewable by the naked eye, but may be revealed when the light source is circularly polarized using a quarter-waveplate in front on the light source, and/or the reflected light passes a quarter-waveplate that is disposed in front of the optical reader (e.g., a camera or other imaging devices). This use of a circularly polarized code has the added benefit that any tampering with the container 100 is even more difficult to hide from the healthcare practitioner, since the patient may not be able to see the circularly polarized code, and may not even understand that a hidden code (i.e., the circularly polarized code) is present on the container 100. In some embodiments, the healthcare practitioner may use a mobile device which is equipped with one or more quarter wave plates disposed in front of its camera and light source. The mobile device may include a software which detects the hidden code disposed on the surface of the container 100 and determines whether or not the container 100 has been tampered with (e.g., based on whether the mobile device can read the combined optical pattern 116 or not).

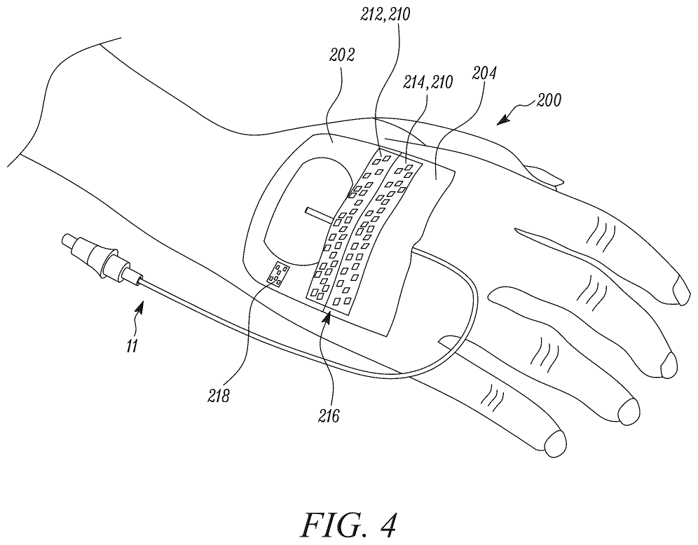

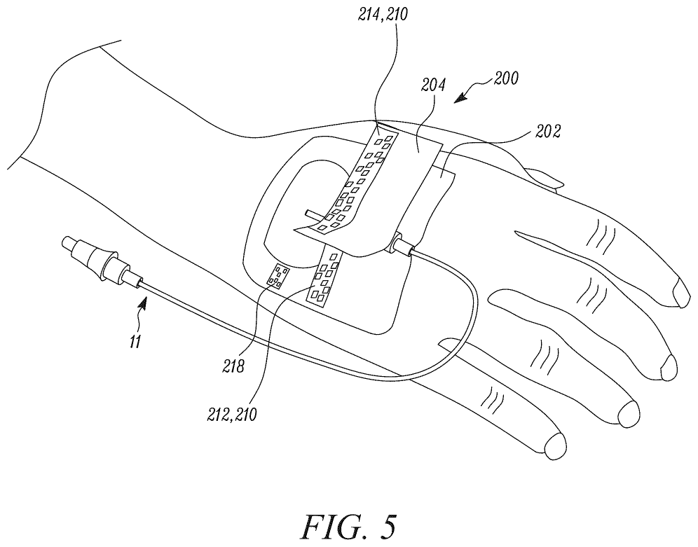

FIGS. 4 and 5 illustrate a wound dressing assembly 200 according to an embodiment of the present disclosure. The wound dressing assembly 200 may be for use with a medical device 11. Specifically, the wound dressing assembly 200 may be for securing the medical device 11 to a skin of a patient. The medical device 11 may include various components such as, for example, a needle, a needle hub, or tubing.

Referring to FIGS. 4 and 5, the wound dressing assembly 200 includes a wound dressing 202 and a cover 204 configured to be attached to the wound dressing 202. The cover 204 may be attached to the wound dressing 202 by any suitable technique, such as via an adhesive. The wound dressing assembly 200 further includes multiple optical patterns 210. The multiple optical patterns 210 are disposed on at least one of the wound dressing 202 or the cover 204. In some embodiments, the multiple optical patterns 210 may be circularly polarized codes (as discussed above with reference to FIGS. 1 to 3).

When the cover 204 is correctly attached to the wound dressing 202, the multiple optical patterns 210 form a combined optical pattern 216 (shown in FIG. 4) that is readable by the optical reader to obtain a first information. When the cover 204 is incorrectly attached to or detached from the wound dressing 202, the multiple optical patterns 210 are unreadable by the optical reader. Each of the multiple optical patterns 210 may be unreadable by the optical reader by itself. For example, when the cover 204 is incorrectly attached to or detached from the wound dressing 202, the multiple optical patterns 210 may be misaligned or spaced apart from each other and may not form the combined optical pattern 216. Therefore, the optical reader may fail to obtain the first information encoded by the combined optical pattern 216 (shown in FIG. 4) when the cover 204 is incorrectly attached to or detached from the wound dressing 202.

The wound dressing assembly 200 may facilitate determining whether the cover 204 is positioned correctly on the wound dressing 202. For example, a user or a healthcare practitioner may attach the cover 204 to the wound dressing 202 and perform a scan of the wound dressing assembly 200 using the optical reader. The optical reader obtaining the first information (i.e., successfully reading the combined optical pattern 216) may indicate that the cover 204 is positioned correctly on the wound dressing 202. However, the optical reader failing to obtain the first information may indicate that the cover 204 is positioned incorrectly on the wound dressing 202, and consequently, may require repositioning or replacement. This may be important as the cover 204 being improperly positioned on the wound dressing 202 may cause discomfort to the patient, and in some cases, may lead to skin injuries.

In the illustrated embodiment of FIGS. 4 and 5, the multiple optical patterns 210 include a first optical pattern 212 disposed on the wound dressing 202 that is unreadable by the optical reader by itself. The multiple optical patterns 210 further include a second optical pattern 214 disposed on the cover 204 that is unreadable by the optical reader by itself. When the cover 204 is correctly attached to the wound dressing 202, the first optical pattern 212 and the second optical pattern 214 may together form the combined optical pattern 216 that is readable by the optical reader to obtain the first information. Further, when the cover 204 is incorrectly attached to or detached from the wound dressing 202, the first optical pattern 212 and the second optical pattern 214 may be misaligned. Thus, when the cover 204 is incorrectly attached to or detached from the wound dressing 202, the first optical pattern 212 and the second optical pattern 214 may not form the combined optical pattern 216, and the optical reader may fail to obtain the first information encoded by the combined optical pattern 216.

In some embodiments, the wound dressing assembly 200 may further include an auxiliary optical pattern 218 disposed on the wound dressing 202. The auxiliary optical pattern 218 may be formed using oxygen-sensitive compounds. For example, the auxiliary optical pattern 218 may be formed by porphyrin molecules. The auxiliary optical pattern 218 may be responsive to oxygen conditions of the skin underneath the wound dressing 202. For example, the auxiliary optical pattern 218 may become visible (e.g., to the naked eye) when the oxygen level under the wound dressing 202 is below a predetermined level. In another example, the auxiliary optical pattern 218 may change color based on the oxygen level under the wound dressing 202. This may alert the healthcare practitioner or the user to change or replace the wound dressing 202 when required. The auxiliary optical pattern 218 may or may not encode an auxiliary information. In some embodiments, the auxiliary optical pattern 218 may be readable by the optical reader to obtain the auxiliary information.

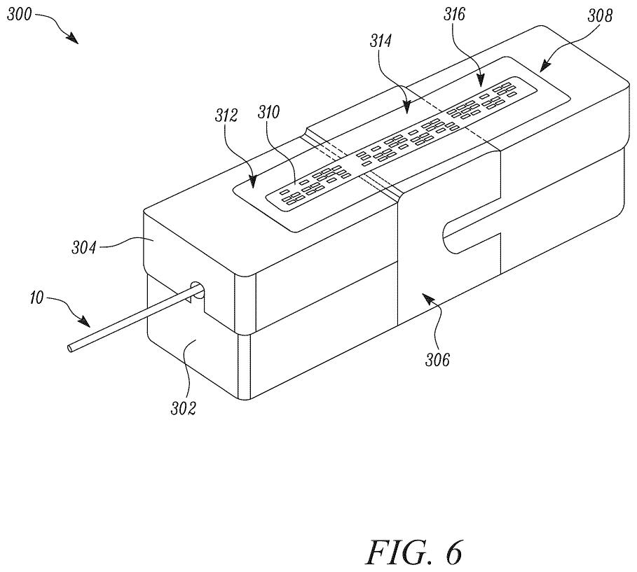

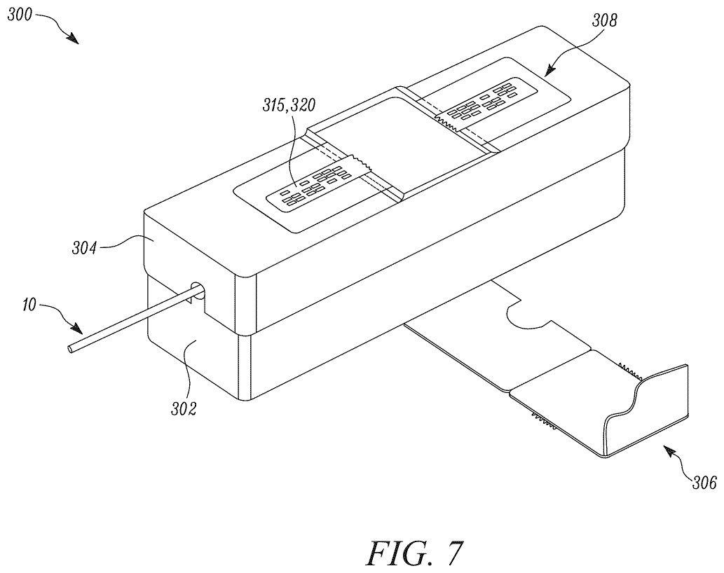

FIGS. 6 and 7 illustrate a container 300 for the medical device 10 according to another embodiment of the present disclosure.

Referring to FIGS. 6 and 7, the container 300 includes a base 302 for holding the medical device 10. The container 300 further includes a cover 304 configured to be coupled with the base 302. The container 300 further includes a locking piece 306 for locking the cover 204 to the base 302. The container 300 further includes an optical pattern 310 disposed or disposable on at least one of the base 302, the cover 304, or the locking piece 306. For example, the optical pattern 310 may be disposed on the locking piece 306 and the cover 304 when the container 300 is closed (i.e., the cover 304 is coupled with the base 302) and the locking piece 306 is locked. The optical pattern 310 encodes a first information.

When the container 300 is closed and the locking piece 306 is locked (as shown in FIG. 6), the optical pattern 310 is readable by an optical reader to obtain the first information. When the container 300 is opened and unlocked (as shown in FIG. 7) after being closed and locked, the optical pattern 310 is modified to a modified optical pattern 315. In FIG. 7, the container 300 is depicted as closed (i.e., the cover 304 is coupled to the base 302) and the locking piece 306 is unlocked. The modified optical pattern 315 has at least one characteristic different from the optical pattern 310. The at least one characteristic of the modified optical pattern 315 that is different from the optical pattern 310 may facilitate detecting tampering with the container 300.

In some embodiments, when the container 300 is closed and the locking piece 306 is locked, a tamper-resistant tape 308 including the optical pattern 310 may be attachable to the cover 304 and the locking piece 306. The tamper-resistant tape 308 may include a first cover portion 312, a second cover portion 316, and a locking piece portion 314 disposed between the first cover portion 312 and the second cover portion 316. The locking piece portion 314 may be at least partially disposable on the locking piece 306. The optical pattern 310 may be at least partially disposed on the locking piece portion 314. Further, when the container 300 is opened and unlocked (shown in FIG. 7) after being closed and locked, the modified optical pattern 315 may be a disfigured optical pattern 320. Specifically, the locking piece 306 may tear away at least a portion of the optical pattern 310, or more specifically, the tamper-resistant tape 308 when the container 300 is opened and unlocked after being closed and locked, such that the optical pattern 310 is modified to the disfigured optical pattern 320. The disfigured optical pattern 320 may be unreadable by the optical reader.

The optical reader may be used to quickly and accurately detect whether the container 300 has been tampered with. The optical reader obtaining the first information (i.e., successfully reading the optical pattern 310) may indicate that the container 300 is closed and the locking piece 306 is locked. This may indicate that the container 300 has not been tampered with. In contrast, the optical reader failing to obtain the first information may indicate that the container 300 has been tampered with. The container 300 may therefore function as a tamper evident device.

When the container 300 is opened and unlocked after being closed and locked, the optical pattern 310 may be disfigured, such that the disfigured optical pattern 320 (shown in FIG. 7) is unreadable by the optical reader. In other words, upon tampering (for example, uncoupling the locking piece 306 from the cover 304), the optical pattern 310 may be disfigured and unreadable by the optical reader. The tamper-resistant tape 308 may also become disfigured consequent to an attempt to tamper with the container 300 by uncoupling the locking piece 306 from the cover 304. The optical pattern 310 may not be re-constructable from the disfigured optical pattern 320.

In some embodiments, the optical pattern 310 may be a circularly polarized code (as discussed above with reference to FIGS. 1 to 3). In some embodiments, the circularly polarized code may be patterned onto a sticker which is placed across the locking piece 306 of the container 300. Specifically, the circularly polarized code may be patterned onto the tamper-resistant tape 308. The circularly polarized code may be disposed on the first cover portion 312, the second cover portion 316, and the locking piece portion 314 of the tamper-resistant tape 308.

The container 300 may also facilitate documenting a series of events during a treatment of a patient. For example, a healthcare practitioner (e.g., a nurse, a medical assistant, a doctor, other clinicians, etc.) may secure the medical device 10 within the container 300. Subsequently, the healthcare practitioner may attach the tamper-resistant tape 308 to the cover 304 and the locking piece 306. Then, the healthcare practitioner may perform a first scan of the container 300 (or more specifically, the tamper-resistant tape 308) using the optical reader. Various initial record information, such as an image of the patient, an image of the container 300, the date, the time, and the like may be recorded (e.g., in a computing device) subsequent to the first scan. The healthcare practitioner may then proceed with their other duties.

After some time elapses, the healthcare practitioner may return to the patient and perform a second scan of the container 300 (or more specifically, the tamper-resistant tape 308) using the optical reader. Various final record information, such as an image of the patient, an image of the container 300, the date, the time, and the like may be recorded (e.g., in a computing device) subsequent to the second scan.

As discussed above, the disfigured optical pattern 320 (shown in FIG. 7) may be unreadable by the optical reader. The optical reader obtaining the information in the second scan (i.e., successfully reading the optical pattern 310) may indicate that the container 300 was not tampered with, and the patient remained protected in the absence of the healthcare practitioner. The healthcare practitioner may discard the container 300, administer new treatment via the medical device 10, and secure the medical device 10 with another unit of the container 300. However, the optical reader failing to obtain the information in the second scan (i.e., failing to read the optical pattern 310) may indicate tampering with the container 300 in the absence of the healthcare practitioner. Evidence of tampering (e.g., the final record information) may be reported in such cases. The aforementioned steps may be performed in a cycle.

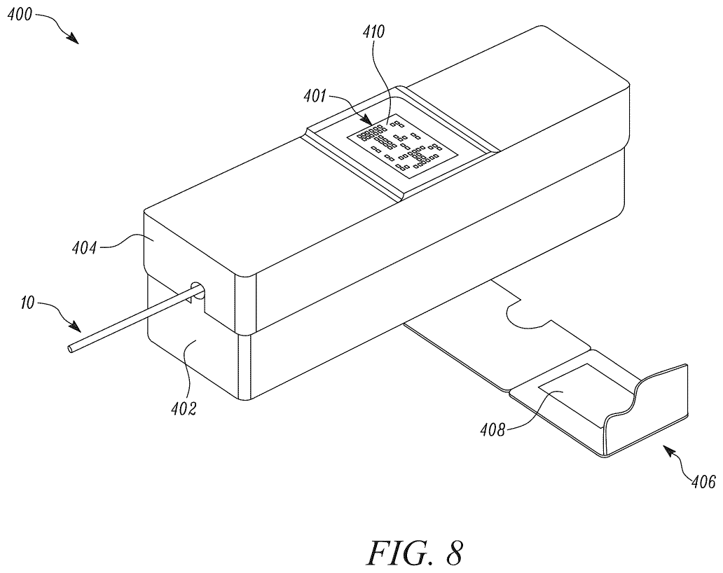

FIGS. 8, 9, and 10 illustrate a container 400 for use with the medical device 10 according to another embodiment of the present disclosure.

The container 400 includes a base 402 for holding the medical device 10. The container 400 further includes a cover 404 configured to be coupled to the base 402. The container 400 further includes a locking piece 406 for locking the cover 404 to the base 402. The container 400 further includes an optical pattern 410 disposed or disposable on at least one of the base 402, the

cover 404, or the locking piece 406. The optical pattern 410 encodes a first information. In the illustrated embodiment of FIGS. 8, 9 and 10, the container 400 further includes an optical modifier 408 at least partially disposed on the locking piece 406.

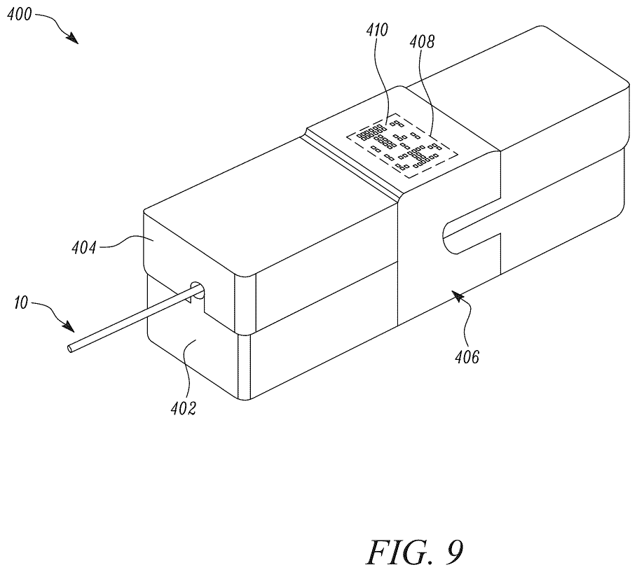

The optical modifier 408 may be configured to interact with the optical pattern 410 to modify at least one characteristic of the optical pattern 410. When the container 400 is closed and the locking piece 406 is locked (as shown in FIG. 9), the optical modifier 408 may interact with the optical pattern 410, such that the optical pattern 410 is readable by an optical reader to obtain the first information.

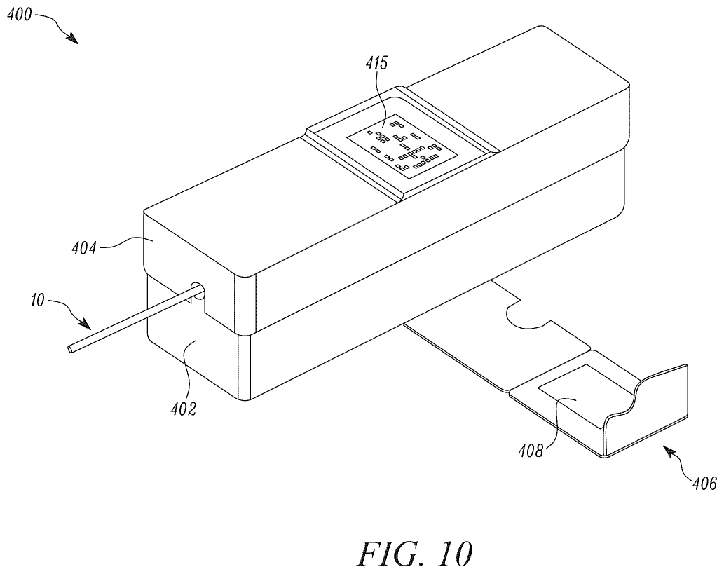

When the container 400 is opened and unlocked (as shown in FIG. 10) after being closed and locked, the optical pattern 410 may be modified to a modified optical pattern 415. In FIG. 10, the container 400 is depicted as closed (i.e., the cover 404 is coupled to the base 402) and the locking piece 406 is unlocked. In some embodiments, when the container 400 is opened and unlocked after being closed and locked, the modified optical pattern 415 may have a different color than that of the optical pattern 410.

The optical modifier 408 may modify the optical pattern 410 to the modified optical pattern 415 when the container 400 is opened and unlocked after being closed and locked. This may facilitate detecting whether the container 400 has been tampered with. Specifically, the modified optical pattern 415 being observable on the container 400 may indicate that the container 400 has been tampered with. In such embodiments, the modified optical pattern 415 may be visually different from the optical pattern 410, such that the modified optical pattern 415 can be distinguished from the optical pattern 410 with naked eye. In some embodiments, the modified optical pattern 415 may have a different color than that of the optical pattern 410. In such embodiments, a healthcare practitioner may detect tampering by observing the color of the modified optical pattern 415.

In some embodiments, the container 400 may further include a multilayered film 401 (shown in FIG. 8) disposed on at least one of the base 402, the cover 404, or the locking piece 406. The optical pattern 410 may be disposed on a layer of the multilayered film 401. In the illustrated embodiment of FIG. 8, the multilayered film 401 and the optical pattern 410 are disposed on the cover 404. Further, the optical modifier 408 may include an adhesive configured to attach with the multilayered film 401 when the container 400 is closed and the locking piece 406 is locked, such that when the container 400 is opened and unlocked after being closed and locked, at least one layer of the multilayered film 401 is removed from the rest of the multilayered film 401 and the optical pattern 410 is modified to the modified optical pattern 415.

In some other embodiments, the optical modifier 408 may include a chemical agent (e.g., phenolphthalein, or porphyrin molecule, among others) configured to interact with the optical pattern 410 when the container 400 is closed and the locking piece 406 is locked, such that a color of at least a portion of the optical pattern 410 changes when the optical pattern 410 is exposed to air (or otherwise exposed to oxygen). Thus, in such embodiments, when the container 400 is opened and unlocked after being closed and locked, the color of at least the portion of the optical pattern 410 is changed.

In some embodiments, the modified optical pattern 415 may be unreadable by the optical reader. In such embodiments, the optical reader failing to obtain the first information (i.e., failing to read the optical pattern 410) may indicate that the container 400 has been tampered with. Thus, the container 400 may function as a tamper evident device.

In some other embodiments, when the container 400 is opened and unlocked after being closed and locked, the modified optical pattern 415 may encode a second information different from the first information, such that the modified optical pattern 415 is readable by the optical reader to obtain the second information. In such embodiments, the optical reader obtaining the second information (i.e., successfully reading the modified optical pattern 415) may indicate that the container 400 has been tampered with.

The container 400 may also facilitate documenting a series of events during a treatment of a patient. For example, a healthcare practitioner (e.g., a nurse, a medical assistant, a doctor, other clinician, etc.) may secure the medical device 10 within the container 400. Subsequently, the healthcare practitioner may perform a first scan of the container 400 using the optical reader. Various initial record information, such as an image of the patient, an image of the container 400, the date, the time, and the like may be recorded (e.g., in a computing device) subsequent to the first scan. The healthcare practitioner may then proceed with their other duties.

After some time elapses, the healthcare practitioner may return to the patient and perform a second scan of the container 400 using the optical reader. Various final record information, such as an image of the patient, an image of the container 400, the date, the time, and the like may be recorded (e.g., in a computing device) subsequent to the second scan.

As discussed above, the optical pattern 410 may be modified to the modified optical pattern 415. The optical reader obtaining the first information in the second scan (i.e., successfully reading the optical pattern 410) may indicate that the container 400 was not tampered with, and the patient remained protected in the absence of the healthcare practitioner. The healthcare practitioner may discard the container 400, administer new treatment via the medical device 10, and secure the medical device 10 with another unit of the container 400. However, the optical reader failing to obtain the first information in the second scan may indicate tampering with the container 400 in the absence of the healthcare practitioner. This may occur due to modification of the optical pattern 410 to the modified optical pattern 415. Evidence of tampering (e.g., the final record information) may be reported in such cases. The aforementioned steps may be performed in a cycle.

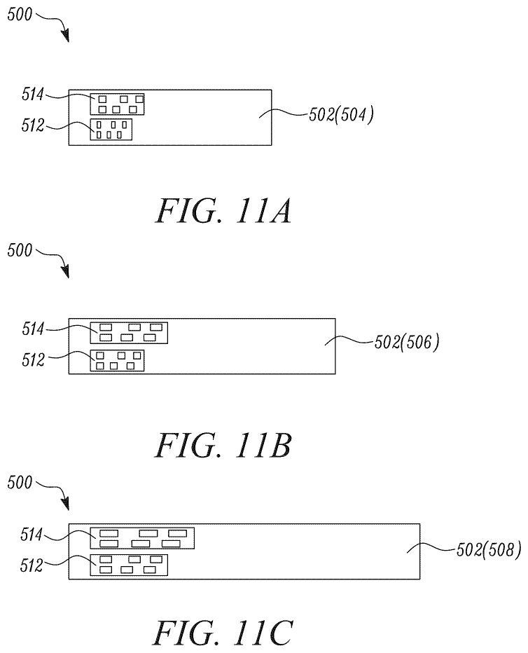

FIGS. 11A, 11B, and 11C illustrate a wound dressing 500 according to an embodiment of the present disclosure.

Referring to FIGS. 11A to 11C, the wound dressing 500 includes a stretchable substrate 502. The stretchable substrate 502 includes an under-tensioned state 504 (shown in FIG. 11A), a correctly-tensioned state 506 (shown in FIG. 11B), and an over-tensioned state 508 (shown in FIG. 11C). The stretchable substrate 502 is configured to be transitioned between the under-tensioned state 504, the correctly-tensioned state 506, and the over-tensioned state 508. The wound dressing 500 may be designed to be used in the correctly-tensioned state 506 of the stretchable substrate 502. It may be noted that the correctly-tensioned state 506 encompasses a plurality of tensioned states at which the wound dressing 500 is designed to be used.

The wound dressing 500 further includes a first optical pattern 512 disposed on the stretchable substrate 502. The first optical pattern 512 encodes a first information. The first optical pattern 512 is readable by the optical reader in the correctly-tensioned state 506 of the stretchable substrate 502 to obtain the first information. The first optical pattern 512 is unreadable by the optical reader in each of the under-tensioned state 504 and the over-tensioned state 508 of the stretchable substrate 502.

The wound dressing 500 may facilitate determining whether the stretchable substrate 502 is in the correctly-tensioned state 506. The wound dressing 500 may be conveniently scanned using the optical reader. The optical reader obtaining the first information (i.e., successfully reading the first optical pattern 512) may indicate that the stretchable substrate 502 is in the correctly-tensioned state 506.

In some embodiments, the wound dressing 500 may further include a second optical pattern 514 disposed on the stretchable substrate 502 and spaced apart from the first optical pattern 512. The second optical pattern 514 may encode a second information different from the first information. The second optical pattern 514 may be readable by the optical reader in the under-tensioned state 504 to obtain the second information. The second optical pattern 514 may be unreadable by the optical reader in the correctly-tensioned state 506. The second optical pattern 514 may be unreadable by the optical reader in the over-tensioned state 508. That is, the second optical pattern 514 may be unreadable by the optical reader in each of the correctly-tensioned state 506 and the over-tensioned state 508.

The wound dressing 500 including the first optical pattern 512 and the second optical pattern 514 may facilitate determining whether the stretchable substrate 502 is in the under-tensioned state 504, the correctly-tensioned state 506, or the over-tensioned state 508. The optical reader obtaining the second information (i.e., successfully reading the second optical pattern 514) may indicate that the stretchable substrate 502 is in the under-tensioned state 504. The optical reader obtaining the first information (i.e., successfully reading the first optical pattern 512) may indicate that the stretchable substrate 502 is in the correctly-tensioned state 506. The optical reader failing to obtain both the first information and the second information (i.e., failing to read each of the first optical pattern 512 and the second optical pattern 514) may indicate that the stretchable substrate 502 is in the over-tensioned state 508.

The stretchable substrate 502 may be configured to be applied to a skin of a patient. The wound dressing 500 may include an intravenous securement dressing, a self-adherent wrap, an elastic bandage, and other medical dressings. A non-limiting example of a self-adherent wrap is the Solventum Corporation COBAN™ Self-Adherent Wrap.

Unless otherwise indicated, all numbers expressing feature sizes, amounts, and physical properties used in the specification and claims are to be understood as being modified by the term “about.” Accordingly, unless indicated to the contrary, the numerical parameters set forth in the foregoing specification and attached claims are approximations that can vary depending upon the

desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

What is claimed is:

1. A container for a medical device, the container comprising:
a base for holding the medical device;
a cover configured to be coupled with the base;
a locking piece for locking the cover to the base; and
multiple optical patterns disposed on at least one of the base, the cover, or the locking piece;
wherein, when the container is closed and the locking piece is locked, the multiple optical patterns form a combined optical pattern readable by an optical reader to obtain a first information, and
wherein, when the container is opened and unlocked after being closed and locked, the multiple optical patterns are unreadable by the optical reader.

2. The container of claim 1, wherein the multiple optical patterns comprise:
a first optical pattern disposed on the cover that is unreadable by the optical reader by itself; and
a second optical pattern disposed on the locking piece that is unreadable by the optical reader by itself;
wherein, when the container is closed and the locking piece is locked, the first optical pattern and the second optical pattern together form the combined optical pattern that is readable by the optical reader to obtain the first information; and
wherein, when the container is opened and unlocked after being closed and locked, the first optical pattern and the second optical pattern are misaligned.

3. A wound dressing assembly, the wound dressing assembly comprising:
a wound dressing;
a cover configured to be attached to the wound dressing;
multiple optical patterns disposed on at least one of the wound dressing or the cover;
wherein, when the cover is correctly attached to the wound dressing, the multiple optical patterns form a combined optical pattern that is readable by an optical reader to obtain a first information, and
wherein, when the cover is incorrectly attached to or detached from the wound dressing, the multiple optical patterns are unreadable by the optical reader.

4. The wound dressing assembly of claim 3, wherein the multiple optical patterns comprise:
a first optical pattern disposed on the wound dressing that is unreadable by the optical reader by itself; and
a second optical pattern disposed on the cover that is unreadable by the optical reader by itself;
wherein, when the cover is correctly attached to the wound dressing, the first optical pattern and the second optical pattern together form the combined optical pattern that is readable by the optical reader to obtain the first information, and
wherein, when the cover is incorrectly attached to or detached from the wound dressing, the first optical pattern and the second optical pattern are misaligned.

5. A container for a medical device, the container comprising:
a base for holding the medical device;
a cover configured to be coupled to the base;
a locking piece for locking the cover to the base; and
an optical pattern disposed or disposable on at least one of the base, the cover, or the locking piece, wherein the optical pattern encodes a first information;
wherein, when the container is closed and the locking piece is locked, the optical pattern is readable by an optical reader to obtain the first information, and wherein, when the container is opened and unlocked after being closed and locked, the optical pattern is modified to a modified optical pattern, the modified optical pattern having at least one characteristic different from the optical pattern.

6. The container of claim 5, when the container is closed and the locking piece is locked, a tamper-resistant tape including the optical pattern is attachable to the cover and the locking piece, the tamper-resistant tape comprising a first cover portion, a second cover portion, and a locking piece portion disposed between the first cover portion and the second cover portion and at least partially disposable on the locking piece, wherein the optical pattern is at least partially disposed on the locking piece portion.

7. The container of claim 6, wherein, when the container is opened and unlocked after being closed and locked, the modified optical pattern is a disfigured optical pattern, and wherein the disfigured optical pattern is unreadable by the optical reader.

8. The container of claim 5, further comprising an optical modifier at least partially disposed on the locking piece and configured to interact with the optical pattern to modify at least one characteristic of the optical pattern, wherein, when the container is closed and the locking piece is locked, the optical modifier interacts with the optical pattern, such that the optical pattern is readable by the optical reader to obtain the first information, and wherein, when the container is opened and unlocked after being closed and locked, the modified optical pattern has a different color than that of the optical pattern.

9. The container of claim 8, wherein the modified optical pattern is unreadable by the optical reader.

10. The container of claim 8, further comprising an optical modifier at least partially disposed on the locking piece and configured to interact with the optical pattern to modify at least one characteristic of the optical pattern, wherein, when the container is closed and the locking piece is locked, the optical modifier interacts with the optical pattern, such that the optical pattern is readable by the optical reader to obtain the first information, and wherein, when the container is opened and unlocked after being closed and locked, the modified optical pattern encodes a second information different from the first information, such that the modified optical pattern is readable by the optical reader to obtain the second information.

11. The container of claim 8, further comprising a multilayered film disposed on at least one of the base, the cover, or the locking piece, wherein the optical pattern is disposed on a layer of the multilayered film, wherein the optical modifier comprises an adhesive configured to attach with the multilayered film when the container is closed and the locking piece is locked, such that when the container is opened and unlocked after being closed and locked, at least one layer of the multilayered film is removed from the multilayered film and the optical pattern is modified to the modified optical pattern.

12. The container of claim 8, wherein the optical modifier comprises a chemical agent configured to interact with the optical pattern when the container is closed and the locking piece is locked, such that a color of at least a portion of the optical pattern changes when the optical pattern is exposed to air, wherein, when the container is opened and unlocked after being closed and locked, the color of at least the portion of the optical pattern is changed.

13. A wound dressing comprising:
a stretchable substrate comprising an under-tensioned state, a correctly-tensioned state, and an over-tensioned state, the stretchable substrate being configured to be transitioned between the under-tensioned state, the correctly-tensioned state, and the over-tensioned state; and
a first optical pattern disposed on the stretchable substrate and encoding a first information, wherein the first optical pattern is readable by an optical reader in the correctly-tensioned state of the stretchable substrate to obtain the first information, and wherein the first optical pattern is unreadable by the optical reader in each of the under-tensioned state and the over-tensioned state of the stretchable substrate.

14. The wound dressing of claim 13, further comprising a second optical pattern disposed on the stretchable substrate and spaced apart from the first optical pattern, the second optical pattern encoding a second information, wherein, the second optical pattern is:
readable by the optical reader in the under-tensioned state to obtain the second information;
unreadable by the optical reader in the correctly-tensioned state; and
unreadable by the optical reader in the over-tensioned state.
